(19) United States
(12) Patent Application Publication
(10) Pub. No.: US 2021/0386611 A1
(43) Pub. Date: Dec. 16, 2021

(54) CLOUD-BASED CONTROL SYSTEM AND METHOD ENABLING INTERACTIVE CLINICAL CARE USING A POWERED MOBILITY ASSISTANCE DEVICE

(71) Applicant: Parker-Hannifin Corporation, Cleveland, OH (US)

(72) Inventors: Skyler A. DALLEY, Shaker Heights, OH (US); Ryan J. FARRIS, Solon, OH (US)

(21) Appl. No.: 17/268,798

(22) PCT Filed: Sep. 3, 2019
(86) PCT No.: PCT/US2019/049284

§ 371 Date: Feb. 16, 2021

Related U.S. Application Data
(60) Provisional application No. 62/773,396, filed on Nov. 30, 2018.

(51) Int. Cl. A61H 3/00; G16H 40/40; G16H 40/67; G16H 20/30; G05B 15/02

(52) U.S. Cl. CPC A61H 3/00; G16H 40/40; G16H 40/67; A61H 2205/10; G05B 15/02; A61H 2201/5012; A61H 2201/5097; G16H 20/30

(57) ABSTRACT

A cloud-based control method and related system implements enhanced control of a network of mobility assistance devices. The control method implemented by the cloud-based system includes the steps of: monitoring performance of the mobility assistance device with a device user electronic device; based on said monitoring, transmitting raw data regarding performance of the mobility assistance device from the device user electronic device to a remote datacenter; processing the raw data with the remote datacenter to generate processed user data that is suitable for use by a data user; transmitting the processed user data to a data user electronic device that is accessible to the data user, wherein the data user generates user actions data for optimizing performance of the mobility assistance device; transmitting the user actions data from a data user electronic device to the remote datacenter; processing the user actions data with the remote datacenter to generate processed control data for optimizing performance of the mobility assistance device; and transmitting the processed control data to a device user electronic device, wherein the processed control data controls performance of the mobility assistance device to optimize performance of the mobility assistance device.

### RELATED APPLICATIONS

This application claims the benefit of U.S. Provisional Application No. 62/773,396 filed Nov. 30, 2018, the contents of which are incorporated herein by reference.

### FIELD OF INVENTION

The present invention relates to electronic control systems for a mobility assistance device, such as for example a legged mobility device or “exoskeleton” device, including data and control systems for optimizing the operation of mobility assistance devices.

### BACKGROUND OF THE INVENTION

There are currently on the order of several hundred thousand spinal cord injured (SCI) individuals in the United States, with roughly 12,000 new injuries sustained each year at an average age of injury of 40.2 years. Of these, approximately 44% (approximately 5300 cases per year) result in paraplegia. One of the most significant impairments resulting from paraplegia is the loss of mobility, particularly given the relatively young age at which such injuries occur. Surveys of users with paraplegia indicate that mobility concerns are among the most prevalent, and that chief among mobility desires is the ability to walk and stand. In addition to impaired mobility, the inability to stand and walk entails severe physiological effects, including muscular atrophy, loss of bone mineral content, frequent skin breakdown problems, increased incidence of urinary tract infection, muscle spasticity, impaired lymphatic and vascular circulation, impaired digestive operation, and reduced respiratory and cardiovascular capacities. In addition to full paraplegia, debilitative health conditions, like strokes and other vascular and neurological impairment, can substantially impair mobility and have the additional secondary physiological effects.

In an effort to restore some degree of legged mobility to individuals with paraplegia or other forms of mobility impairment, several lower limb orthoses have been developed. The simplest form of such devices is passive orthotics with long-leg braces that incorporate a pair of ankle-foot orthoses (AFOs) to provide support at the ankles, which are coupled with leg braces that lock the knee joints in full extension. The hips are typically stabilized by the tension in the ligaments and musculature on the anterior aspect of the pelvis. Since almost all energy for movement is provided by the upper body, these passive orthoses require considerable upper body strength and a high level of physical exertion, and provide very slow walking speeds.

The hip guidance orthosis (HGO), which is a variation on long-leg braces, incorporates hip joints that rigidly resist hip adduction and abduction, and rigid shoe plates that provide increased center of gravity elevation at toe-off, thus enabling a greater degree of forward progression per stride. Another variation on the long-leg orthosis, the reciprocating gait orthosis (RGO), incorporates a kinematic constraint that links hip flexion of one leg with hip extension of the other, typically by means of a push-pull cable assembly. As with other passive orthoses, the user leans forward against a stability aid (e.g., bracing crutches or a walker) while un-weighting the swing leg and utilizing gravity to provide hip extension of the stance leg. Since motion of the hip joints is reciprocally coupled through the reciprocating mechanism, the gravity-induced hip extension also provides contralateral hip flexion (of the swing leg), such that the stride length of gait is increased. One variation on the RGO incorporates a hydraulic-circuit-based variable coupling between the left and right hip joints. Experiments with this variation indicate improved hip kinematics with the modulated hydraulic coupling.

To decrease the high level of exertion associated with passive orthoses, the use of powered orthoses has been under development, which incorporate actuators and drive motors associated with a power supply to assist with locomotion. These powered orthoses have been shown to increase gait speed and decrease compensatory motions, relative to walking without powered assistance.

The use of powered orthoses presents an opportunity for electronic control of the orthoses. Exoskeleton devices to date, however, have lacked comprehensive control systems that also are user-friendly to maximize the effectiveness and comfort for a legged mobility exoskeleton device. Examples of powered orthoses are known. WO/2010/044087, US 2010/0094188, and U.S. Pat. No. 8,096,965 disclose a powered exoskeleton bracing system/exoskeleton bracing system. These prior art devices, however, have been insufficient for comprehensive and user-friendly control of the exoskeleton device. There have been attempts to provide at least generalized control of an exoskeleton device, including the providing of safety indications. For example, U.S. Pat. No. 8,905,955 B2 discloses a method of controlling an exoskeleton bracing system comprising halting actuation of the motorized joints when a signal that is received from a tilt sensor indicates falling. These methods are described entirely within the context of standing and sitting transitions.

WO/2013/142777 discloses a method of controlling a powered lower extremity orthotic, wherein the leg support includes a thigh segment, shank segment, further comprising estimating an angle of the shank segment with respect to vertical. The device is control to take a step when the shank angle exceeds a threshold with respect to gravity, and the system further comprises signaling the user when placing the orthotic into a state corresponding to taking a step, the signal generally being accomplished by an auditory tone, haptic vibration, or visual cue. WO/2013/142777 also discloses a related method of controlling a powered lower extremity orthotic, wherein the leg support includes a thigh segment, shank segment. The method comprises estimating an angle of the shank segment with respect to vertical, and the device takes a step when the shank angle exceeds a threshold with respect to gravity. The method further comprises calculating a center of pressure average trajectory over time, calculating the variation of that location over time, and generating a proficiency score. The method further comprises restricting which exoskeleton states may be reached based on at least a threshold of said amount of variation.

WO/2014/159577 discloses a lower extremity orthosis configured to be coupled to a person, and a controller that receives signals from a plurality of sensors. The controller estimates at least one feedback ready value based on the sensor output, and at least one feedback system operated by the controller is configured to communicate the feedback ready value to the user. The orthosis provides the user with orthosis operational information not otherwise available to the user, wherein the feedback systems include at least one light indicating actuator effort, a plurality of lights proportionally indicating actuator torque, at least one light indicating force at an interface point, a plurality of lights proportionally indicating force at an interface point. The feedback ready value is selected from: force between user and orthosis, effort applied by orthosis, torque applied by orthosis, maximum effort applied over gait cycle, average effort applied over gait cycle, center of pressure, limb position, center of mass position, foot clearance, orthosis state, next orthosis action, optimal gain aid orientation, and movement of the person.

Although the above conventional control systems provide a certain level of control, the scope of control has been limited. Such control systems for exoskeleton devices and other mobility assistance devices to date have lacked comprehensiveness in a manner that is user-friendly to maximize the effectiveness for enhanced user performance and physical improvement. In addition, it would be desirable to share performance data among multiple interested parties, including patients and device users, clinicians, and device developers and manufactures. Shared information regarding broader usage and experiences could aid in enhanced performance for individual users. For example, access to treatment experiences and histories, technological advances, device analytics, and other information that can optimize device performance and user capability may be limited in certain geographical locations relative to others. Accordingly, under-served communities could benefit from performance information from more experienced personnel. Such sharing of information, however, has not been readily achieved in conventional control systems. Accordingly, current exoskeleton devices and other powered mobility assistance devices are not being utilized to their full potential.

### SUMMARY OF THE INVENTION

The present invention is directed to cloud-based control systems and methods that enable interactive clinical care using a powered lower extremity orthosis or exoskeleton device. Embodiments of the disclosed control systems and methods enable clinicians to remotely interact with a mobility or rehabilitation assistance device, such as for example a powered exoskeleton device, to provide a continuum of care that benefits from historical data of an individual user, and/or aggregated inter-user data of multiple users, for the purpose of setting device parameters or user goals, and analyzing other related performance metrics for optimization of device usage.

While conventional mobility assistance device control systems have begun to use cloud-based technology for device analytics, the control systems and methods of the present disclosure allow for cloud-based device interaction, providing a deeper level of remote care that benefits from the analytical data that cloud-based solutions can provide. Device use can be monitored and adjusted in a convenient and timely manner, enhancing both device safety and usability. This approach extends the continuum of care beyond the local clinic and into the broader community, and also increases the consistency of care as emergent treatments or treatment results can be shared among clinics and clinicians.

Embodiments of the present invention include a cloud-based data center, which includes a plurality of Internet-connected servers, in which information from a rehabilitation or mobility device may be received, stored, and processed. This data, also referred to herein as “device user outcomes” data, may be accessed by and presented to a third party (e.g. a clinician), who then may act on the data to enhance performance with another device. Such clinician or third-party responses, also referred to herein as “user actions data”, may be employed to provide peer results information, device alteration such as changing device settings, and the like to affect device behavior and user treatment. By recording and associating device user outcomes and user actions data, the data center enables other data users, and particularly those who are inexperienced, to make more informed decisions about how to properly implement device behavior and user treatment.

In exemplary embodiments, data gathered regarding device use by device users (e.g., patient, clinician), referred to herein as “raw data”, is transferred either directly such as from the device itself, or indirectly such as via a connected mobile device or local computer, to the data center where the raw data is stored. The raw data received at the data center is then initially processed by the data center prior to or following storage, such that the raw data may be presented in useful form to or acted upon another party, referred to herein as a “data user”. The data user, for example, may be another patient, clinician, researcher, or other third party, who may receive the processed user data on a local computing device, such as for example a mobile device or local computer. The data users may use the processed user data to generate the referenced user actions data, such as for example altering or recommending device settings, altering or recommending device usage, providing feedback to the device user, and the like.

Data that is gathered as a result of generating the user actions data is then secondarily processed by the data center into processed control data, such that the control data may be presented in useful form for further action and analysis by the mobility assistance device and device users. The processed control data is then transferred to the mobility assistance device and/or electronic control devices associated with the mobility assistance device, and once received, the processed control data is utilized to optimize use of the mobility assistance device by device users. The cloud-based system of the present invention thereby provides a feedback loop, by which information regarding device use may be received from and shared by multiple sources, be further acted upon in a variety of ways, and used to implement and optimize device use. This transformational approach thereby provides more information about how device behavior or user treatment has changed or may need to change, and further provides options for how and when these changes can be implemented.

The cloud-based system, therefore, provides broad based sharing of information including access to treatment experiences and histories of others, technological advances that occur in any location, device analytics of multiple devices, and other information that can optimize device performance and user capability that otherwise would be subjected to limited access. Accordingly, under-served communities could benefit from performance information from more experienced personnel by the cloud-based sharing of information. Benefits of described systems and methods include, for example, tracking patient progress, setting patient goals, enabling device functions, enforcing usage restrictions, providing progressive therapy, active device monitoring, and the like. Another advantage is the provision of “crowd-sourced” clinical care, in which clinicians having little device exposure or a smaller user base can draw insight from the actions of clinics with more device experience and larger user populations.

An aspect of the invention, therefore, is a control method and related cloud-based control system for implementing enhanced cloud-based control of a network of mobility assistance devices. In exemplary embodiments, the control method includes the steps of: monitoring performance of the mobility assistance device with a device user electronic device; based on said monitoring, transmitting raw data regarding performance of the mobility assistance device from the device user electronic device to a remote datacenter; processing the raw data with the remote datacenter to generate processed user data that is suitable for use by a data user; transmitting the processed user data to a data user electronic device that is accessible to the data user, wherein the data user generates user actions data for optimizing performance of the mobility assistance device; transmitting the user actions data from a data user electronic device to the remote datacenter; processing the user actions data with the remote datacenter to generate processed control data for optimizing performance of the mobility assistance device; and transmitting the processed control data to a device user electronic device, wherein the processed control data controls performance of the mobility assistance device to optimize performance of the mobility assistance device.

The control method may include transmitting raw data comprising user outcomes data pertaining to each of a plurality of mobility assistance devices to the remote datacenter, wherein the remote datacenter aggregates the device user outcomes data as to the plurality of mobility assistance devices to generate the processed user data. The processed user data further may be transmitted to a plurality of data user electronic devices, wherein one or more of the plurality of data users generates user actions data for optimizing performance of the mobility assistance device. The control method further may include transmitting user actions data from a plurality of data user electronic devices to the remote datacenter, wherein the remote datacenter aggregates the user actions data from the plurality of data users to generate the processed control data. The processed control data further may be transmitted to a plurality of device user electronic devices, wherein one or more of the plurality of device users uses the processed control data to control performance of the mobility assistance device. By aggregating and distributing the processed user data and processed control data across multiple networked devices, the benefits of crowd sourced clinical care are achieved.

Another aspect of the invention is a cloud-based control system for controlling a mobility assistance device by performing the control method according to any of the embodiments. Another aspect of the invention is a non-transitory computer readable medium that stores executable program code, which when executed by a computer performs the control method according to any of the embodiments, and the program code in particular is executed by one or more components of the cloud-based control system.

These and further features of the present invention will be apparent with reference to the following description and attached drawings. In the description and drawings, particular embodiments of the invention have been disclosed in detail as being indicative of some of the ways in which the principles of the invention may be employed, but it is understood that the invention is not limited correspondingly in scope. Rather, the invention includes all changes, modifications and equivalents coming within the spirit and terms of the claims appended hereto. Features that are described and/or illustrated with respect to one embodiment may be used in the same way or in a similar way in one or more other embodiments and/or in combination with or instead of the features of the other embodiments.

### BRIEF DESCRIPTION OF THE DRAWINGS

FIG. 1 is a drawing depicting an exemplary exoskeleton device as being worn by a user.

FIG. 2 is a drawing depicting a perspective view of an exemplary exoskeleton device in a standing position.

FIG. 3 is a drawing depicting a perspective view of the exemplary exoskeleton device in a seated position.

FIG. 4 is a drawing depicting a front view of the exemplary exoskeleton device in a standing position.

FIG. 5 is a drawing depicting a side view of the exemplary exoskeleton device in a standing position.

FIG. 6 is a drawing depicting a back view of the exemplary exoskeleton device in a standing position.

FIG. 7 is a drawing depicting a schematic block diagram of operative portions of an exemplary control system and related electronic components of a mobility assistance device in accordance with embodiments of the present invention.

FIG. 8 is a drawing depicting an exemplary cloud-based architecture for operation of a system of mobility assistance devices in accordance with embodiments of the present invention.

FIG. 9 is a drawing depicting an exemplary data flow across the cloud-based architecture of FIG. 8.

FIG. 10 is a drawing depicting an exemplary data flow for cloud-based analytics using the cloud-based architecture of FIG. 8.

FIG. 11 is a drawing depicting an exemplary data flow for cloud-based control using the cloud-based architecture of FIG. 8.

### DETAILED DESCRIPTION

Embodiments of the present invention will now be described with reference to the drawings, wherein like reference numerals are used to refer to like elements throughout. It will be understood that the figures are not necessarily to scale.

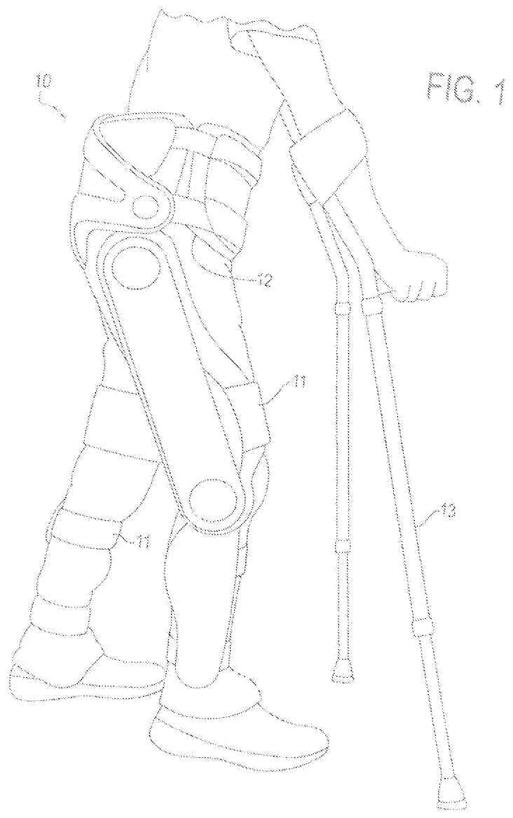

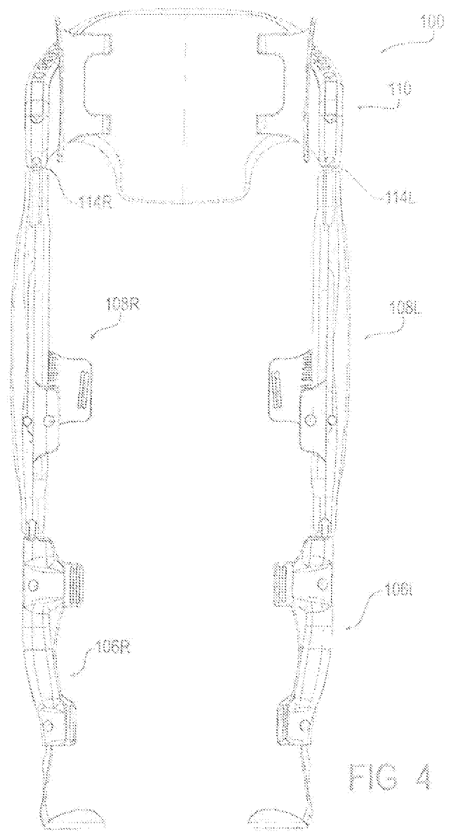

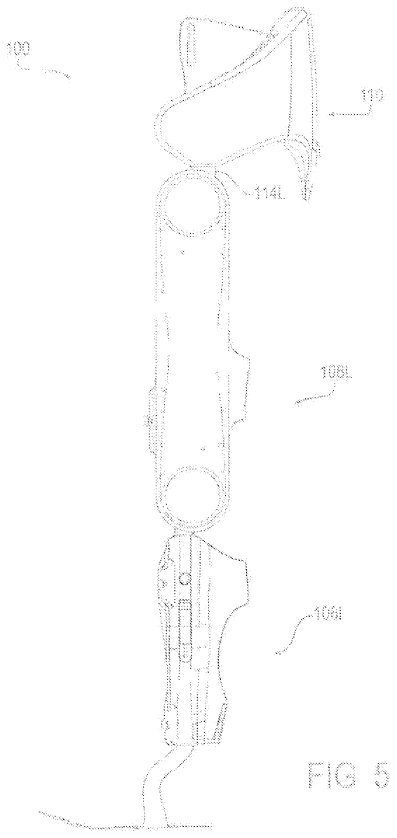

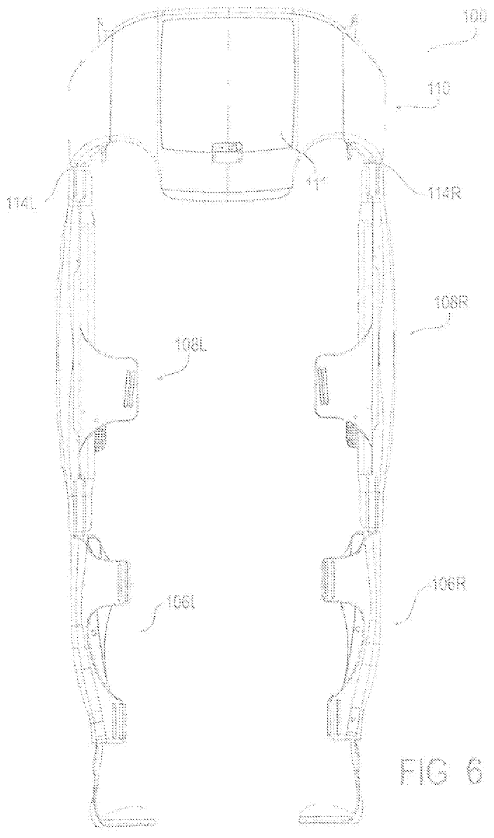

For context, FIGS. 1-6 depict various views of an exemplary exoskeleton device that may be used in connection with the control system and methods of the present invention. A somewhat generalized description of such exoskeleton device is provided here for illustration purposes. A more detailed description of such device may be found in Applicant's International Patent Appl. No. PCT/US2015/023624 filed on Mar. 3, 2015, which is incorporated here in its entirety by reference. It will be appreciated, however, that the described exoskeleton device presents an example usage, and that the control system and methods of the present invention are not limited to any particular configuration of an exoskeleton device. Variations may be made to the exoskeleton device, while the features of the present invention remain applicable. In addition, the principles of this invention may be applied generally to any suitable mobility device. Such mobility devices include, for example, orthotic devices which aid in mobility for persons without use or limited use of a certain body portion, and prosthetic devices, which essentially provide an electro-mechanical replacement of a body part that is not present such as may be used by an amputee or a person congenitally missing a body portion.

As show in FIG. 1, an exoskeleton device 10, which also may be referred to in the art as a “wearable robotic device”, can be worn by a user. To attach the device to the user, the device 10 can include attachment devices 11 for attachment of the device to the user via belts, loops, straps, or the like. Furthermore, for comfort of the user, the device 10 can include padding 12 disposed along any surface likely to come into contact with the user. The device 10 can be used with a stability aid 13, such as crutches, a walker, or the like.

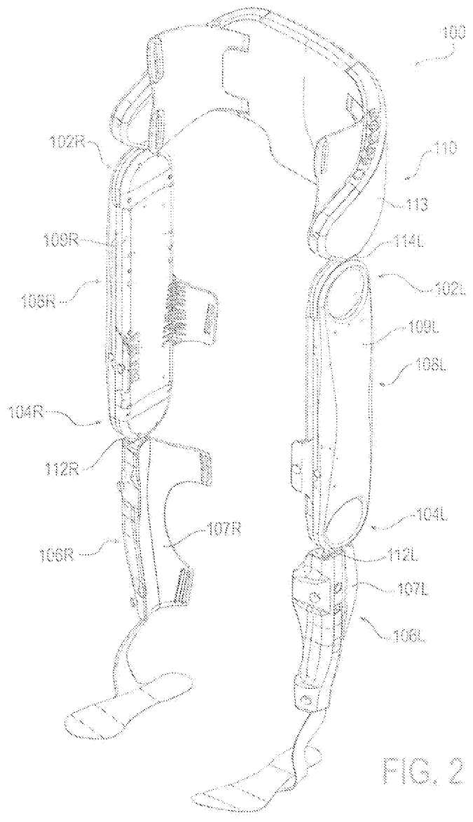

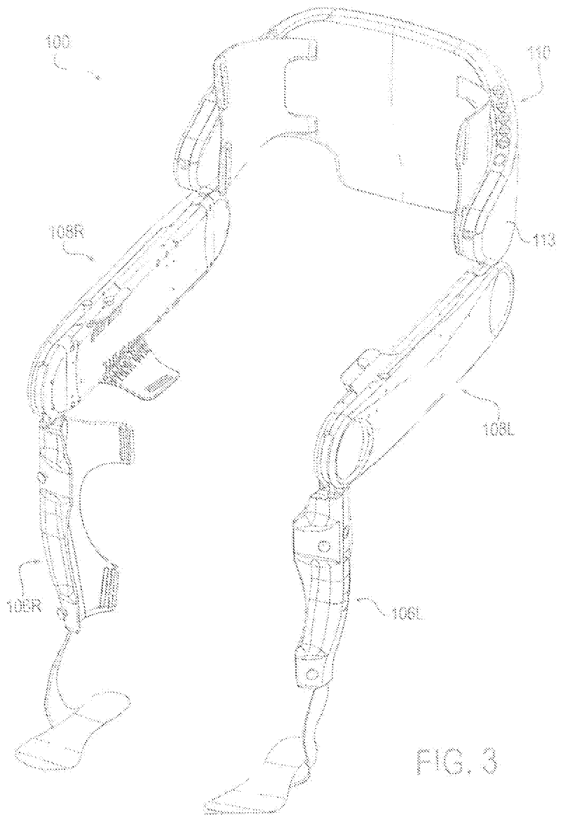

An exemplary legged mobility exoskeleton device is illustrated as a powered lower limb orthosis 100 in FIGS. 2-6. Specifically, the orthosis 100 shown in FIGS. 2-6 may incorporate four drive components configured as electro-motive devices (for example, electric motors), which impose sagittal plane torques at each knee and hip joint components including (right and left) hip joint components 102R, 102L and knee joint components 104R, 104L. FIG. 2 shows the orthosis 100 in a standing position while FIG. 3 shows the orthosis 100 in a seated position.

As seen in the figures, the orthosis contains five assemblies or modules, although one or more of these modules may be omitted and further modules may be added (for example, arm modules), which are: two lower (right and left) leg assemblies (modules) 106R and 106L, two (left and right) thigh assemblies 108R and 108L, and one hip assembly 110. Each thigh assembly 108R and 108L includes a respective thigh assembly housing 109R and 109L, and link, connector, or coupler 112R and 112L extending from each of the knee joints 104R and 104L and configured for moving in accordance with the operation of the knee joints 104R and 104L to provide sagittal plane torque at the knee joints 104R and 104L.

The connectors 112R and 112L further may be configured for releasably mechanically coupling each of thigh assembly 108R and 108L to respective ones of the lower leg assemblies 106R and 106L. Furthermore, each thigh assembly 108R and 108L also includes a link, connector, or coupler 114R and 114L, respectively, extending from each of the hip joint components 102R and 102L and moving in accordance with the operation of the hip joint components 102R and 102L to provide sagittal plane torque at the knee joint components 104R and 104L. The connectors 114R and 114L further may be configured for releasably mechanically coupling each of thigh assemblies 108R and 108L to the hip assembly 110.

In some embodiments, the various components of device 100 can be dimensioned for the user. However, in other embodiments the components can be configured to accommodate a variety of users. For example, in some embodiments one or more extension elements can be disposed between the lower leg assemblies 106R and 106L and the thigh assemblies 108R and 108L to accommodate users with longer limbs. In other configurations, the lengths of the two lower leg assemblies 106R and 106L, two thigh assemblies 108R and 108L, and one hip assembly 110 can be adjustable. That is, thigh assembly housings 109R, 109L, the lower leg assembly housings 107R and 107L for the lower leg assemblies 106R, 106L, respectively, and the hip assembly housing 113 for the hip assembly 110 can be configured to allow the user or medical professional to adjust the length of these components in the field. For example, these components can include slidable or movable sections that can be held in one or more positions using screws, clips, or any other types of fasteners. In view of the foregoing, the two lower leg assemblies 106R and 106L, two thigh assemblies 108R and 108L, and one hip assembly 110 can form a modular system allowing for one or more of the components of the orthosis 100 to be selectively replaced and for allowing an orthosis to be created for a user without requiring customized components. Such modularity can also greatly facilitate the procedure for donning and doffing the device.

In orthosis 100, each thigh assembly housing 109R, 109L may include substantially all the drive components for operating and driving corresponding ones of the knee joint components 104R, 104L and the hip joint components 102R, 102L. In particular, each of thigh assembly housings 109R, 109L may include drive components configured as two motive devices (e.g., electric motors) which are used to drive the hip and knee joint component articulations. However, the various embodiments are not limited in this regard, and some drive components can be located in the hip assembly 110 and/or the lower leg assemblies 106R, 106L.

A battery 111 for providing power to the orthosis can be located within hip assembly housing 113 and connectors 114R and 114L can also provide means for connecting the battery 111 to any drive components within either of thigh assemblies 108R and 108L. For example, the connectors 114R and 114L can include wires, contacts, or any other types of electrical elements for electrically connecting battery 111 to electrically powered components in thigh assemblies 108R and 108L. In the various embodiments, the placement of battery 111 is not limited to being within hip assembly housing 113. Rather, the battery can be one or more batteries located within any of the assemblies of orthosis 100.

The referenced drive components may incorporate suitable sensors and related internal electronic controller or control devices for use in control of the exoskeleton device. Such internal control devices may perform using the sensory information the detection of postural cues, by which the internal control device will automatically cause the exoskeleton device to enter generalized modes of operation, such as sitting, standing, walking, variable assist operation, and transitions between these generalized modes or states (e.g., Sit to Stand, Stand to Walk, Walk to Stand, Stand to Sit, etc.) and step transition (e.g., Right Step, Left Step). The internal electronic control devices further may perform fall mitigation and recovery operations for the exoskeleton device, as described for example in Applicant's International Patent Appl. No. PCT/US2016/016319 filed on Feb. 3, 2016, which is incorporated here in its entirety by reference.

The internal electronic control devices and related electronics further may incorporate or include a mobility assistance device communications interface that is configured to transmit and receive signals over an electronic signal interface. In exemplary embodiments, the mobility device communications interface may communicate electronically over a wireless interface by transmitting signals to and receiving signals from a communications interface of an electronic communication device including a control application for controlling the drive components of the mobility device.

To perform such operations, the drive systems and internal control device of the mobility assistance device may employ the use of accelerometers, gyroscopes, inertial measurement, and other sensors to detect and observe the upper leg orientation or angle and angular velocity. The internal control device may then selectively control the drive components to modulate the joint components, and particularly the knee and hip joint components, to apply torque, implement locked or released states, or otherwise effect positioning or movement of the joint components control of the exoskeleton device for mode operation or for fall mitigation.

To implement the features of the present invention, the exoskeleton device or other mobility device may include a control system having one or more processor devices that are configured to execute program code stored on a non-transitory computer readable medium embodying the control methods associated the generalized control of the exoskeleton device, including the control operations of the present invention. It will be apparent to a person having ordinary skill in the art of computer programming of electronic devices how to program the electronic control device to operate and carry out logical functions associated with present invention. Accordingly, details as to specific programming code have been left out for the sake of brevity. Also, controller functionality could be carried out via dedicated hardware, firmware, software, or any combinations thereof, without departing from the scope of the invention. As will be understood by one of ordinary skill in the art, therefore, the control system may have various implementations. For example, the control system may be configured as any suitable processor device, such as a programmable circuit, integrated circuit, memory and I/O circuits, an application specific integrated circuit, microcontroller, complex programmable logic device, other programmable circuits, or the like. The control system may also include a non-transitory computer readable medium, such as random access memory (RAM), a read-only memory (ROM), an erasable programmable read-only memory (EPROM or Flash memory), or any other suitable medium. Instructions for performing the methods described below may be stored in the non-transitory computer readable medium and executed by the processor device.

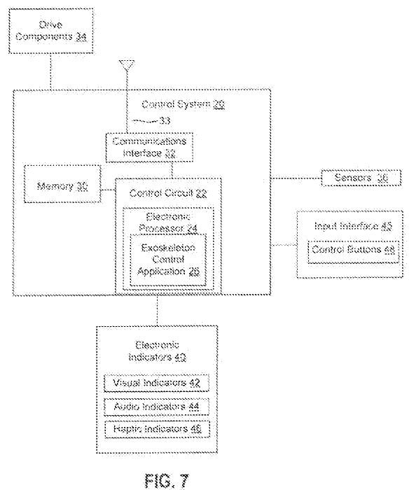

FIG. 7 is a drawing depicting a schematic block diagram of operative portions of an exemplary control system 20 and related electronic components in accordance with embodiments of the present invention, that is a component of the mobility assistance device such as the exoskeleton device of the previous figures. The control system 20 may include a primary control circuit 22 that is configured to carry out various control operations relating to control of the exoskeleton device. The control circuit 22 may include an electronic processor 24, such as a CPU, microcontroller or microprocessor. Among their functions, to implement the features of the present invention, the control circuit 22 and/or electronic processor 24 may comprise an electronic controller that may execute program code embodied as the exoskeleton control application 26. It will be apparent to a person having ordinary skill in the art of computer programming, and specifically in application programming for electronic and communication devices, how to program the device to operate and carry out logical functions associated with application 26. Accordingly, details as to specific programming code have been left out for the sake of brevity.

The exoskeleton control application 26 may be stored in a non-transitory computer readable medium, such as random access memory (RAM), a read-only memory (ROM), an erasable programmable read-only memory (EPROM or Flash memory), or any other suitable medium. In the example of FIG. 7, the exoskeleton control application 26 is shown as being stored internally within the processing components, but the application also may be stored in an additional memory device such as the memory 30. Instructions for performing device control that are stored in the non-transitory computer readable medium may be executed by the processor components 22 and 24. Also, while the code may be executed by control circuit 22 or processor 24 in accordance with an exemplary embodiment, such controller functionality could also be carried out via dedicated hardware, firmware, software, or combinations thereof, without departing from the scope of the invention.

The control system 20 may constitute internal electronic control devices and related electronics incorporated into one or more of the exoskeleton device components, and typically may be incorporated into one or more of the thigh assembly or hip assembly. The control system 20 further may include a communications interface 32 for electronic communication with components external to the control system. For example, the communications interface may provide for electronic communication via an antenna 33 with an external mobile communication device, and thus may be configured to transmit and receive signals over an electronic signal interface. In exemplary embodiments, the communications interface may communicate electronically with an external mobile communication device over a wireless interface by transmitting signals to and receiving signals from the drive components for control of the mobility device. A mobile communications device and related control systems and methods are disclosed Applicant's International Patent Appl. No. PCT/US2016/40304 filed on Jun. 30, 2016, which is incorporated here in its entirety by reference.

The control system 20 further may be in electronic communication with both sensory and drive components of the exoskeleton device. The connections may be hard wired connections via internal circuit boards and other wired connections, but wireless communication also may be employed between the control system and/or sensor and drive components. In FIG. 7 the drive components are generally indicated by block 34, and the sensors are generally indicated by block 36. For gathering appropriate sensory information, the sensors 36 may include the use of accelerometers, gyroscopes, inertial measurement, and other sensors to detect and observe the upper leg and torso orientation or angle and angular velocity. Example sensors may include hall effect sensors, magnetic angle sensors, accelerometer sensors, gyroscope sensors, resistance temperature detectors, and others. There also may be one or more redundant sensors that correspond respectively to one or more of the above sensors, and the redundant sensors may provide sensor information when there is a sensor fault detected in a respective sensor.

The control system 20 may then selectively control the drive components 34 to configure and modulate the joint components of the exoskeleton device, and particularly the knee and hip joint components, to apply torque, implement locked or released states, or otherwise effect positioning or movement of the joint components control of the exoskeleton device for various modes of operation and for fall mitigation.

As described for example in Applicant's referenced previous patent applications, in the described exoskeleton device operation generally is automated based on sensory detections. As an example, to go from sit to stand a user may pull in the legs and lean forward, as any person normally does when getting ready to stand. Upon sensing such a pre-standing position, the exoskeleton drive system would send a haptic feedback signal to the user, such as a vibration indicator, informing the user that a transition to standing will occur. Control of mobility mode of operation (sit, stand, walk, etc.), and transitions between mobility modes, proceeds as warranted. Mode transitions and mode operation, therefore, is operated generally by the sensors reading user postural cues, which are interpreted by the control system that in turn generates control signals to drive operation of the drive components.

The control system 20 further may be in electronic communication with a plurality of electronic indicators 40. In FIG. 7, the electronic indicators are generally indicated by block 40. The electronic indicators may include visual indicators 42 that indicate aspects of device state and operation by lighting. In exemplary embodiments, the lighting may be color-coded lighting in which light emitting diodes (LEDs) are employed as the visual indicators. The electronic indicators further may include audio indicators 44, by which speakers may be employed to provide audio alerts pertaining to aspects of device state and operation. Different sounds may be employed for different types of audio alerts, and may be used in combination with the visual indicators 42 to provide multiple indicator combinations corresponding to information pertaining to different aspects of device state and operation. The electronic indicators further may include haptic indicators 46. The haptic indicators 46 may be configured as vibration generators that provide vibration indications as alerts pertaining to aspects of device state and operation.

The control system 20 further may be in electronic communication with an input interface 45. The input interface may be configured as an electronic control panel or comparable control interface that permits user inputs for control of the exoskeleton device. The input or control interface 45 may include and one or more control inputs 48 that may provide a varied array of control options for a user, including a power button for turning on an enabling the exoskeleton device.

The present invention is directed to cloud-based control systems and control methods that enable interactive clinical care using a powered lower extremity orthosis or exoskeleton device, such as the device described above. Again, the principles of this invention may be applied generally to any suitable mobility assistance device, including powered orthotic devices and powered prosthetic devices. Embodiments of the disclosed control systems and methods enable clinicians to remotely interact with such a mobility or rehabilitation assistance device to provide a continuum of care that benefits from historical data of an individual user, and/or aggregated inter-user data of multiple users, for the purpose of setting device parameters or user goals for optimal performance.

While conventional systems have begun to use cloud-based technology for device analytics, the control systems and methods of the present disclosure allow for cloud-based device interaction, providing a deeper level of remote care that benefits from the analytical data that cloud-based solutions can provide. Device use can be monitored and adjusted in a convenient and timely manner, enhancing both device safety and usability. This approach extends the continuum of care beyond the local clinic and into the broader community, and also increases the consistency of care as emergent treatments or treatment results can be shared among clinics and clinicians.

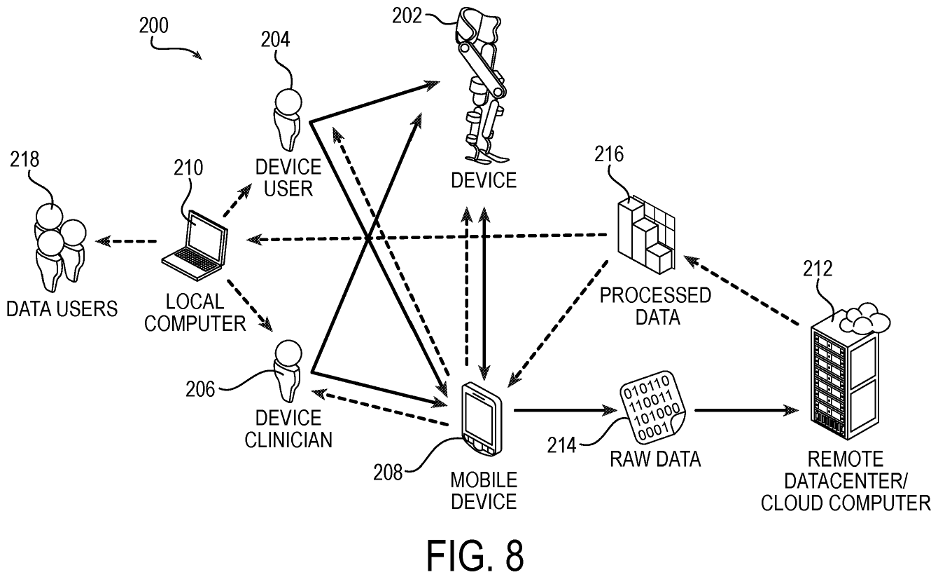

FIG. 8 is a drawing depicting an exemplary cloud-based architecture 200 for operation of a system of mobility assistance devices. FIG. 8 depicts generally the participants in such a system, with the arrows representing potential lines of electronic communication that may be employed for data transfers, control commands, and physical usage. Individual system components are shown for simplicity, but it will be appreciated that multiple components of each type may be networked in a cloud-based system of devices and users performing different roles.

The exemplary architecture 200 includes a mobility assistance device 202, illustrated here as an exoskeleton device as a suitable example, that is used by a device user 204 who uses the device 202 for mobility assistance. In this context, a device user may be a patient who is directly using the device for mobility assistance such as the device user 204, or another person 206 who may be studying, operating, evaluating, or otherwise using the device outside of mobility assistance. For example, device user 206 identified in FIG. 8 may be a device clinician 206 who may be a healthcare professional or similar device operator with expertise in the performance and use of the mobility assistance device 202. The device clinician 206 will work with the device user 204 to optimize performance of the mobility assistance device 202 to maximize the user experience with the device 202 to improve overall health and to reach user mobility performance goals. As referenced above, although FIG. 8 depicts a single mobility assistance device 202, device user 204, and device clinician 206, it will be appreciated that the architecture 200 will include any suitable number of such participants, and indeed as many as practicable, to provide a cloud-connected “community” of participants who may be located anywhere and share information as an electronic network as further detailed below.

To provide such a cloud-connected network, the architecture 200 includes a plurality of electronic devices that may be used by device users and/or device clinicians. For example, the electronic devices may include mobile devices 208 and other computing devices like local computers 210. Mobile devices may be more suitable for use in the field to monitor and control performance of the mobility assistance device 202, whereas local computers may be more suited to more rigorous data storage and analysis As referenced above, a communications interface on the mobility assistance device 202 may communicate electronically with the external mobile communication device 208 over a wireless interface by the exchange of electronic signals (again see Applicant's International Patent Appl. No. PCT/US2016/40304). The electronic signals, for example, may include control signals from the mobile device 208 to the mobility assistance device 202 relating to the settings, control, and performance of the mobility assistance device 202. Other control signals may include data transfers between the mobility assistance device 202 and the mobile device 208 relating to the performance and operation of the mobility assistance device 202, which then may be analyzed for optimizing performance of devices across the system as further detailed below.

The architecture 200 further may include a cloud-based computing system, such as one or more network servers or the like, that acts as a remote datacenter 212. The remote datacenter 212 operates for the gathering and analysis of data, and subsequent sharing of information across the system. In general operation, raw data 214 that is gathered by multiple mobile devices 208 is transmitted up to the remote datacenter 212. The datacenter 212 performs requisite analyses and data processing for sharing information across the system, examples of which may include cloud-based data analytics and cloud-based control implementation. Details as to processing for cloud-based data analytics and could-based control are described below. In exemplary embodiments for both such categories of operation, the remote datacenter 212 generates processed data 216 that results from the processing of the raw data 214. The processed data 216 then may be transmitted back to local computers 210 and/or to mobile devices 208. The processed data 216 in turn may be used by device users 204 and/or device clinicians 206 to optimize or otherwise enhance performance of the mobility assistance devices 202. In addition, local computers 210 may be accessed by third-party data users 218 who are otherwise not associated with the device users or clinicians who initially generated the raw data 214. The data users may be different device users and clinicians who are using different mobility assistance devices from a device associated with the original raw data 214, or may be third parties who are merely studying device performance across the cloud-based community of users. In this manner, information regarding multiple mobility assistance devices across the system may be gathered, combined, and shared in a manner that permits a given mobility assistance device user to benefit from the experiences of multiple device users across the system.

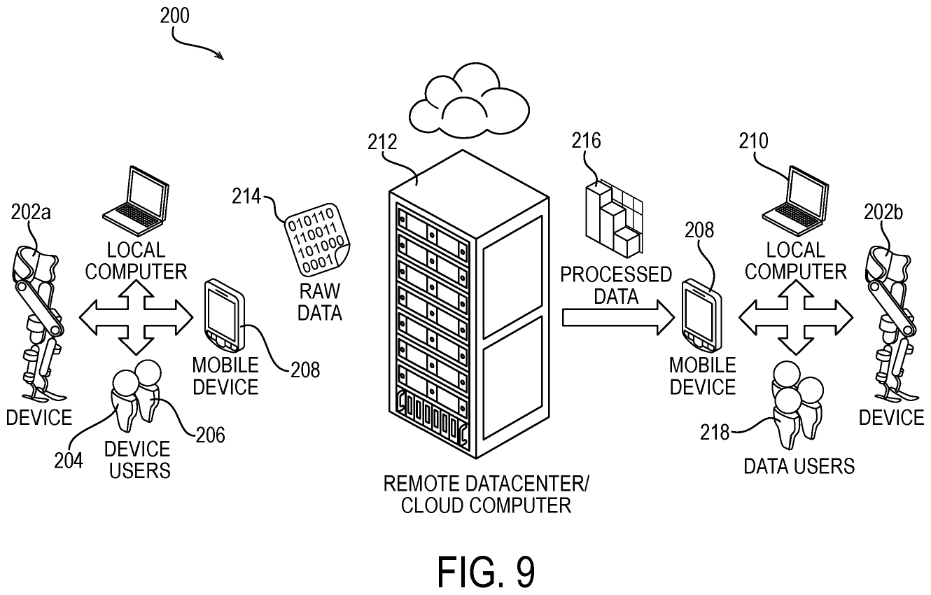

As illustrative of such data and information sharing, FIG. 9 is a drawing depicting an exemplary data flow across the cloud-based architecture 200 of FIG. 8 relating to multiple mobility assistance devices. In the example of FIG. 9, data flows between a first mobility assistance device 202a and a second mobility assistance device 202b via the cloud-based remote datacenter 212, although it will be appreciated that any suitable number of mobility assistance devices and related users and supporting electronic devices may be operating across the architecture 200. Comparably as in FIG. 8, the representation of the exemplary architecture 200 of FIG. 9 includes a first mobility assistance device 202a, illustrated here as an exoskeleton device as a suitable example, that is used by a patient device user 204 who directly uses the device 202a for mobility assistance and that may be operated by a clinician device user 206. Information regarding the mobility assistance device 202a may be received, stored, and processed by the remote or cloud-based datacenter 212 as the raw data 214. Because the raw data 214 uploaded to the remote datacenter 212 originates based on mobility assistance device usage by the device users, this raw data 214 also may be referred to herein as “device user outcomes” in reference to performance of the device user 204 utilizing the mobility assistance device 202a.

The data corresponding to the device user outcomes is then processed by the remote datacenter 212, which as referenced above preferably is a cloud-based computing system. Generally, processed data 216, which is described in more detail below, may be outputted and then accessed by and presented to third party data users 218, such as clinicians or users of other mobility assistance devices such as the second mobility assistance device 202b. The processed data 216 may be received and accessed by any suitable electronic device, such as for example another mobile device 208 or a local computer 210. The third-party data users 218 then may act on the processed data to generate device operations that enhance performance of mobility assistance devices across the system. Such clinician or third-party responses, also referred to herein as “user actions data”, may be employed to develop aggregated device parameters that are based on peer results of multiple users. The user actions data may include, for example, recommendations or implementation commands for parameters relating to device alterations such as changing device settings, and the like to affect device behavior and user performance. By recording and associating device user outcomes and user actions data, the remote datacenter 212 enables other data users, and particularly those who are inexperienced, to make more informed decisions about how to properly implement device behavior for optimizing user treatment and performance.

An aspect of the invention, therefore, is a control method and related control system for implementing enhanced cloud-based control of a network of mobility assistance devices. In exemplary embodiments, the control method includes the steps of: monitoring performance of the mobility assistance device with a device user electronic device; based on said monitoring, transmitting raw data regarding performance of the mobility assistance device from the device user electronic device to a remote datacenter; processing the raw data with the remote datacenter to generate processed user data that is suitable for use by a data user; transmitting the processed user data to a data user electronic device that is accessible to the data user, wherein the data user generates user actions data for optimizing performance of the mobility assistance device; transmitting the user actions data from a data user electronic device to the remote datacenter; processing the user actions data with the remote datacenter to generate processed control data for optimizing performance of the mobility assistance device; and transmitting the processed control data to a device user electronic device, wherein the processed control data controls performance of the mobility assistance device to optimize performance of the mobility assistance device.

The control method may include transmitting raw data comprising user outcomes data pertaining to each of a plurality of mobility assistance devices to the remote datacenter, wherein the remote datacenter aggregates the device user outcomes data as to the plurality of mobility assistance devices to generate the processed user data. The processed user data further may be transmitted to a plurality of data user electronic devices, wherein one or more of the plurality of data users generates user actions data for optimizing performance of the mobility assistance device. The control method further may include transmitting user actions data from a plurality of data user electronic devices to the remote datacenter, wherein the remote datacenter aggregates the user actions data from the plurality of data users to generate the processed control data. The processed control data further may be transmitted to a plurality of device user electronic devices, wherein one or more of the plurality of device users uses the processed control data to control performance of the mobility assistance device. By aggregating and distributing the processed user data and processed control data across multiple networked devices, the benefits of crowd sourced clinical care is achieved whereby clinicians having little device exposure or a smaller user base can draw insight from the actions of clinicians with more device experience and larger user populations.

Another aspect of the invention is a cloud-based control system for controlling a mobility assistance device by performing the control method according to any of the embodiments. Another aspect of the invention is a non-transitory computer readable medium that stores executable program code, which when executed by a computer performs the control method according to any of the embodiments, and the program code in particular is executed by one or more components of the cloud-based control system.

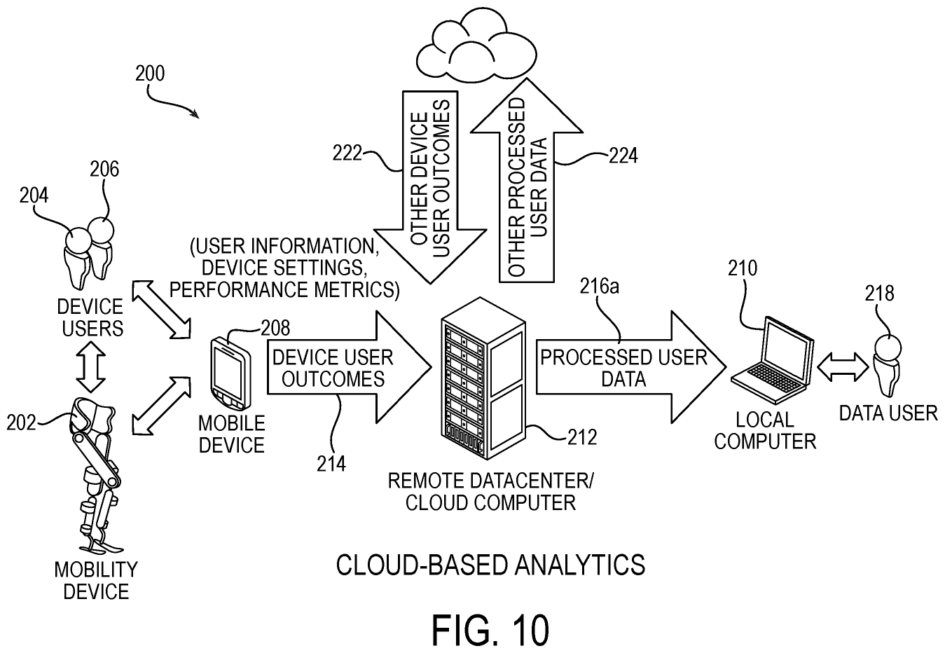

In accordance with such overall system usage, one exemplary usage of the cloud-based system 200 is for cloud-based analytics, by which device user outcomes are gathered across the system for study of device performance by data users. FIG. 10 is a drawing depicting an exemplary data flow for cloud-based analytics. In such example, the mobility assistance device 202 is used by a device user (e.g., direct user or patient 204 and/or clinician 206), with the device usage being monitored by any suitable device user electronic device. In this example, the device user electronic device is a mobile device 208 as the portability of a mobile device renders such device particularly suitable for mobility assistance device monitoring (i.e., a mobile device can be taken wherever a user may be using the mobility assistance device). It will also be appreciated that although FIG. 10 illustrates the device user electronic device as a separate device, the device user electronic device may be a component of the mobility assistance device itself, including for example the control system 20 operating over the communications interface 32 as illustrated in FIG. 7. In this manner, there may be direct communication between the mobility assistance device 202 and the remote datacenter 212. As used herein, therefore, device user electronic devices include both separate electronic devices (e.g., a mobile device like a smartphone or table PC), and electronic devices that are integrated into the mobility assistance device.

Raw data 214 corresponding to device user outcomes is gathered by the monitoring performed by the device user electronic device 208, and the raw data is transmitted to the remote, cloud-based computing datacenter 212. The device user outcomes data may be any data pertaining to device usage of the specific mobility assistance device 202. Such data may include, for example, user information regarding the device user 204/206, device settings of the mobility assistance device 202, performance metrics corresponding to use of the mobility assistance device 202 by the device user 204/206, and like data that corresponds to aspects of usage of the specific mobility assistance device 202.

As seen in FIG. 10, the device user outcomes data transmitted to the remote datacenter 212 is not limited to data corresponding to device user outcomes solely for the mobility assistance device 202. Rather, the remote datacenter 212 further may receive data corresponding to other device user outcomes 222 corresponding to any number of additional mobility assistance devices. In this manner, the remote datacenter 212 receives and aggregates the device user outcomes from multiple mobility assistance devices across the cloud-based system. The remote datacenter 212 can then organize the device user outcomes data and otherwise process such data into a useable form for overall review, analysis, and study. Generally, the raw data of the device user outcomes 214 received at the datacenter is initially processed by the datacenter prior to or following storage, such that the aggregated device user outcome data may be presented in useful form to another party that may act upon such data, i.e., the third-party data user 218. As referenced above, the data user 218, for example, may be another patient, clinician, researcher, or other third party, who may receive the processed user data on a local computing device, such as for example another mobile device or a local computer.

As illustrated in FIG. 10, therefore, processed user data 216a is transmitted to any suitable data user electronic device for analysis or other usage by a data user 218. In this example, the data user electronic device is a local computer 210 as being optimum for data analysis, although any suitable electronic device may be employed as is suitable for the data user's purpose, including another mobile device or an electronic device integrated into another mobility assistance device. Similarly as with the device user outcomes being aggregated from multiple device users, other processed user data 224 may be transmitted to other data user electronic devices of multiple data users across the cloud-based system for a system wide analytical framework. The data users may use the processed user data to generate the referenced user actions data relating to device analytics, such as for example altering or recommending device settings, altering or recommending device usage, providing feedback to the device user, and the like. The cloud-based analytics, therefore, provide for an aggregated presentation of device user outcomes to data users, which then may be employed by the data users to optimize parameters relating to the use of mobility assistance devices.

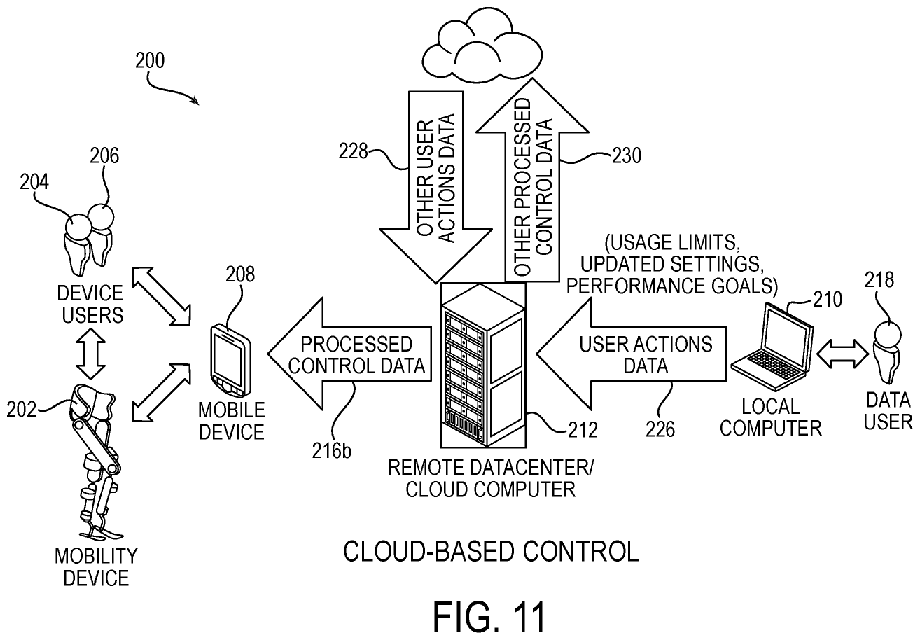

Accordingly, FIG. 11 is a drawing depicting an exemplary data flow for cloud-based control, which is based upon the analysis of the processed user data of FIG. 10 by the data users 218. The data user 218 initially utilizes the data user electronic device 210 (e.g., local computer) to generate a data set 226 corresponding to user actions. Under certain circumstances, the user actions data optionally may relate to a given mobility assistance device of a specific user. As the user actions data 226 is based on processed user data derived from aggregated device user outcomes, however, the user actions data 226 alternatively need not relate to any specific mobility assistance device, but instead may constitute more community-based recommendations or control commands intended for mobility assistance device usage across the system. The user actions data may include recommendations and/or control commands, for example, relating to updated settings for mobility assistance devices, performance goals for users of mobility assistance devices, device usage limits, device trouble shooting or related maintenance information, and like data that may constitute recommendations, control commands, and/or other information generally that may be useful to users of mobility assistance devices across the community of users.

Other user actions data further may include data actions corresponding to opening access to new features, or opening access to advanced features that may be present but have been dormant. Such features may include addition or alteration of performance thresholds, such as for example unlocking increased speed capabilities or use time limits to permit increased usage by a patient. On the other hand, instead of unlocking enhanced performance parameters, restrictive parameters may be incorporated as part of the user actions data. For example, maximum step counts for triggering device service may be monitored, with alerts being sent as to service needs, ultimately resulting in device lock-out for lack of servicing. More in-depth indicators and warnings of device health also may be provided, with device disabling being made accessible as warranted. Activity limits based on environmental conditions, such as temperature for example, also may be implemented. Any related software updates also may be incorporated into the user actions data, which also may include program “bug” controls, patches, software fixes, and the like. Accordingly, the user actions data may constitute data for implementing a wide range of device updates and usage alterations for one or a community of mobility assistance devices.

The user actions data 226 is transmitted by the data user 218 using the data user electronic device 210 to the cloud-based remote datacenter 212. The datacenter 212 then secondarily processes the user actions data 226, such that the user actions data may be presented in useful form for further action and implementation by the mobility assistance devices and/or device users. The data gathered and processed based on the user actions data is referred to herein as processed control data, and the processed control data then is transmitted to the device user side of the system for adapting performance of the mobility assistance device. As shown in FIG. 11, for example, the processed control data 216b may be transmitted the device user electronic device 208 (which may be the same or different from the device that transmits the device user outcomes in FIG. 10). As shown in FIG. 11, the electronic device 208 again may be a mobile device as an example, although any suitable electronic device may be employed. As referenced above, the device user electronic device alternatively may be integrated into the mobility assistance device itself as part of the device control system. Once the processed control data 216b is received via the device user electronic device 208, the processed control data is utilized to optimize use of the mobility assistance device 202 by device users (direct users such as patients 204 and/or clinicians 206).

Similarly as with the data flow for device analytics of FIG. 10, the user actions data transmitted to the remote datacenter 212 is not limited to data corresponding solely to the single data user 218. Rather, the remote datacenter 212 further may receive data corresponding to other user actions 228 corresponding to any number of additional data users. In this manner, the remote datacenter 212 receives and aggregates the user actions data from multiple data users across the system. Likewise, other processed control data 230 may be transmitted to device user electronic devices of multiple device users across the cloud-based system for a system wide device control framework. The device users may use the processed control data to perform the referenced device updates and actions, such as for example altering device settings, altering device usage, analyzing feedback provided to the device user in the processed control data, and the like.

Implementation of the processed control data may be voluntary and thus be implemented by device user action. For example, the device user (e.g., patient 204 or clinician 206) may take action with a mobile device or other remote electronic control device 208, or with a control input interface (see FIG. 7) on the mobility assistance device 202 itself, to set device settings based on recommendations set forth in the processed control data 216b. In another exemplary embodiment, automated responses to the processed control data may be implemented in the mobility assistance device 202 upon satisfaction of one or more predetermined operational conditions. For example, a data user may set or recommend conditions that are incorporated into the processed control data, such that data user actions are triggered automatically in a mobility assistance device in response to device user outcomes (e.g. “when average step speed reaches 0.25 m/s, enable advanced mode settings”, or, “if the device user walks five times in a given week, send positive feedback.”) or some other stimulus (e.g. “send device user reminder to walk every week.”). In this manner, advantageous or optimized usage based on the processed control data may be implemented in the mobility assistance device in a variety of user-implemented and/or automated ways.

The cloud-based system of the present invention thereby provides a feedback loop, by which information regarding device use may be received from and shared by multiple sources, be further acted upon in a variety of ways, and used to implement and optimize device use for multiple users and devices across the system. This transformational approach thereby provides more information about how device behavior or user treatment has changed or may need to change, and further provides options for how and when these changes can be implemented in the mobility assistance devices.

In this manner, the cloud-based system provides broad based sharing of information including access to treatment experiences and histories of others, technological advances that occur in any location, device analytics of multiple devices, and other information that can optimize device performance and user capability that otherwise would be subjected to limited access. Accordingly, under-served communities could benefit from performance information from more experienced personnel by the cloud-based sharing of information. Benefits of the described systems and methods include, for example, tracking patient progress, setting patient goals, enabling device functions, enforcing usage restrictions, providing progressive therapy, active device monitoring, and the like. As referenced above, another advantage is the provision of crowd-sourced clinical care, in which clinicians having little device exposure or a smaller user base can draw insight from the actions of clinicians with more device experience and larger user populations.

In exemplary embodiments, the system may be configured to provide for tailored user accounts, including the capability to disable and enable various mobility assistance device settings or operational parameters based on patient user and/or clinical circumstances. For example, the system may be configured to provide for the following exemplary basic account types: Personal User Accounts for device users of mobility assistance devices, who generally can only access their own Personal User Account; (2) Clinical User Accounts for specific clinicians associated with specific users, who generally may access only their own Clinical User Account and optionally associated Personal User Account of patients under their care to monitor and affect device use; and (3) Therapist Accounts which are more organizational such as for broader clinics, hospitals, and other care centers, by which supervising therapists can access multiple Clinical User Accounts at the therapist's care center organization.

In exemplary configurations, Therapist and Personal User Accounts can either be created by a service provider that acts as a hub for users across the system, such as through an online portal, or requested by a User/Therapists through a mobile computer application such as the mobile applications described above. All accounts initially may be in a disabled state. Once an account is verified, such as by verification of access to a corresponding account email address, the account is enabled by the service provider, and the account can either remain a Personal User Account, or be changed to a Therapist Account and tied to an organization. A therapist with access to a created Therapist Account can also create an account for one or more Clinical Users. Once a Therapist creates a Clinical User Account, such Clinical User Account may be accessible by all therapists within the corresponding organization, although Clinical User Accounts may remain disabled such that clinical users cannot login independently of therapist permission.

An aspect of the invention, therefore, is a control method and related system for implementing enhanced cloud-based control of a network of mobility assistance devices. In exemplary embodiments, the control method includes the steps of: monitoring performance of the mobility assistance device with a device user electronic device; based on said monitoring, transmitting raw data regarding performance of the mobility assistance device from the device user electronic device to a remote datacenter; processing the raw data with the remote datacenter to generate processed user data that is suitable for use by a data user; transmitting the processed user data to a data user electronic device that is accessible to the data user, wherein the data user generates user actions data for optimizing performance of the mobility assistance device; transmitting the user actions data from a data user electronic device to the remote datacenter; processing the user actions data with the remote datacenter to generate processed control data for optimizing performance of the mobility assistance device; and transmitting the processed control data to a device user electronic device, wherein the processed control data controls performance of the mobility assistance device to optimize performance of the mobility assistance device. The control method may include one or more of the following features, either individually or in combination.

In an exemplary embodiment of the control method, the raw data comprises data corresponding to device user outcomes pertaining to device use of a specific mobility assistance device.

In an exemplary embodiment of the control method, the control method further includes transmitting device user outcomes data pertaining to each of a plurality of mobility assistance devices to the remote datacenter, wherein the remote datacenter aggregates the device user outcomes data as to the plurality of mobility assistance devices to generate the processed user data.

In an exemplary embodiment of the control method, the processed user data is transmitted to a plurality of data user electronic devices, wherein one or more of the plurality of data users generates user actions data for optimizing performance of the mobility assistance device.

In an exemplary embodiment of the control method, the device user outcomes data includes one or more of information about the device user, device settings of the mobility assistance device, and performance metrics corresponding to use of the mobility assistance device.

In an exemplary embodiment of the control method, the control method further includes transmitting user actions data from a plurality of data user electronic devices to the remote datacenter, wherein the remote datacenter aggregates the user actions data from the plurality of data users to generate the processed control data.

In an exemplary embodiment of the control method, the processed control data is transmitted to a plurality of device user electronic devices, wherein one or more of the plurality of device users uses the processed control data to control performance of the mobility assistance device.

In an exemplary embodiment of the control method, the user actions data comprises recommendations and/or control commands for optimizing performance of the mobility assistance device.

In an exemplary embodiment of the control method, the user actions data comprises recommendations and/or control commands relating to one or more of updating settings for mobility assistance devices, performance goals for users of mobility assistance devices, device usage limits, and/or device trouble shooting or maintenance information.

In an exemplary embodiment of the control method, the control method further includes implementing the processed control data by device user action applied to a control interface that controls the mobility assistance device.

In an exemplary embodiment of the control method, the control interface is a component of an electronic control device that is remote from the mobility assistance device.

In an exemplary embodiment of the control method, the control interface is a component of the mobility assistance device.

In an exemplary embodiment of the control method, the control method further includes implementing the processed control data automatically upon satisfaction of an operational condition related to the mobility assistance device.

In an exemplary embodiment of the control method, the remote datacenter is a cloud-based computing system.

In an exemplary embodiment of the control method, one or more of the device user electronic device and the data user electronic device is a mobile device.

In an exemplary embodiment of the control method, the device user electronic device is a component of the mobility assistance device.

In an exemplary embodiment of the control method, the mobility assistance device is a legged mobility exoskeleton device.

In an exemplary embodiment of the control method, the device user is a patient direct user and/or a clinician operator of a first mobility assistance device.

In an exemplary embodiment of the control method, the data user is a patient direct user and/or a clinician operator of a second mobility assistance device different from the first mobility assistance device.

Another aspect of the invention is a cloud-based control system for controlling a mobility assistance device by performing the control method according to any of the embodiments. Another aspect of the invention is a non-transitory computer readable medium that stores executable program code, which when executed by a computer performs the control method according to any of the embodiments, and the program code in particular is executed by one or more components of the cloud-based control system.

Although the invention has been shown and described with respect to a certain embodiment or embodiments, it is obvious that equivalent alterations and modifications will occur to others skilled in the art upon the reading and understanding of this specification and the annexed drawings. In particular regard to the various functions performed by the above described elements (components, assemblies, devices, compositions, etc.), the terms (including a reference to a “means”) used to describe such elements are intended to correspond, unless otherwise indicated, to any element which performs the specified function of the described element (i.e., that is functionally equivalent), even though not structurally equivalent to the disclosed structure which performs the function in the herein illustrated exemplary embodiment or embodiments of the invention. In addition, while a particular feature of the invention may have been described above with respect to only one or more of several illustrated embodiments, such feature may be combined with one or more other features of the other embodiments, as may be desired and advantageous for any given or particular application.

1. A control method of performing cloud-based control of a mobility assistance device comprising the steps of:
monitoring performance of the mobility assistance device with a device user electronic device;
based on said monitoring, transmitting raw data regarding performance of the mobility assistance device from the device user electronic device to a remote datacenter;
processing the raw data with the remote datacenter to generate processed user data that is suitable for use by a data user;
transmitting the processed user data to a data user electronic device that is accessible to the data user, wherein the data user generates user actions data for optimizing performance of the mobility assistance device;
transmitting the user actions data from a data user electronic device to the remote datacenter;
processing the user actions data with the remote datacenter to generate processed control data for optimizing performance of the mobility assistance device; and
transmitting the processed control data to a device user electronic device, wherein the processed control data controls performance of the mobility assistance device to optimize performance of the mobility assistance device.

2. The control method of claim 1, wherein the raw data comprises data corresponding to device user outcomes pertaining to device use of a specific mobility assistance device.

3. The control method of claim 2, further comprising transmitting device user outcomes data pertaining to each of a plurality of mobility assistance devices to the remote datacenter, wherein the remote datacenter aggregates the device user outcomes data as to the plurality of mobility assistance devices to generate the processed user data.

4. The control method of claim 2, wherein the processed user data is transmitted to a plurality of data user electronic devices, wherein one or more of the plurality of data users generates user actions data for optimizing performance of the mobility assistance device.

5. The control method of claim 1, wherein the device user outcomes data includes one or more of information about the device user, device settings of the mobility assistance device, and performance metrics corresponding to use of the mobility assistance device.

6. The control method of claim 1, further comprising transmitting user actions data from a plurality of data user electronic devices to the remote datacenter, wherein the remote datacenter aggregates the user actions data from the plurality of data users to generate the processed control data.

7. The control method of claim 1, wherein the processed control data is transmitted to a plurality of device user electronic devices, wherein one or more of the plurality of device users uses the processed control data to control performance of the mobility assistance device.

8. The control method of claim 1, wherein the user actions data comprises recommendations and/or control commands for optimizing performance of the mobility assistance device.

9. The control method of claim 8, wherein the user actions data comprises recommendations and/or control commands relating to one or more of updating settings for mobility assistance devices, performance goals for users of mobility assistance devices, device usage limits, and/or device trouble shooting or maintenance information.

10. The control method of claim 1, further comprising implementing the processed control data by device user action applied to a control interface that controls the mobility assistance device.

11-12. (canceled)

13. The control method of claim 1, further comprising implementing the processed control data automatically upon satisfaction of an operational condition related to the mobility assistance device.

14. The control method of claim 1, wherein the remote datacenter is a cloud-based computing system.

15. The control method of claim 1, wherein one or more of the device user electronic device and the data user electronic device is a mobile device.

16. The control method of claim 1, wherein the device user electronic device is a component of the mobility assistance device.

17. The control method of claim 1, wherein the mobility assistance device is a legged mobility exoskeleton device.

18. The control method of claim 1, wherein the device user is a patient direct user and/or a clinician operator of a first mobility assistance device.

19. The control of method of claim 18, wherein the data user is a patient direct user and/or a clinician operator of a second mobility assistance device different from the first mobility assistance device.

20. A cloud-based control system for controlling a mobility assistance device comprising:
a device user electronic device configured to monitor performance of the mobility assistance device and configured to transmit raw data regarding performance of the mobility assistance device;
remote datacenter configured to receive the raw data from the device user electronic device and configured to process the raw data to generate processed user data that is suitable for use by a data user, and to transmit the processed user data;
a data user electronic device that is accessible to the data user and that receives the processed data, wherein the data user generates user actions data for optimizing performance of the mobility assistance device, and the data user electronic device further is configured to transmit the user actions data to the remote datacenter; and
the remote datacenter further is configured to process the user actions data to generate processed control data for optimizing performance of the mobility assistance device, and to transmit the processed control data to a device user electronic device, wherein the processed control data controls performance of the mobility assistance device to optimize performance of the mobility assistance device.

21. The cloud-based control system of claim 20, wherein the cloud-based control system further is configured to perform the control method of claim 2.

22. A non-transitory computer readable medium that stores executable program code, which when executed by a computer performs the control method of claim 1.
